(19) United States
(12) Patent Application Publication
(10) Pub. No.: US 2014/0090450 A1
(43) Pub. Date: Apr. 3, 2014

(54) Test Rig And Method For Simulating And Analyzing Petrochemical Fouling

(71) Applicants: Denis Richmond Dugwell, London (GB); Colin Paul Hale, London (GB); Geoffrey Frederick Hewitt, Oxfordshire (GB); Sandro Macchietto, London (GB); Jerzy Pental, Reading (GB)

(72) Inventors: Denis Richmond Dugwell, London (GB); Colin Paul Hale, London (GB); Geoffrey Frederick Hewitt, Oxfordshire (GB); Sandro Macchietto, London (GB); Jerzy Pental, Reading (GB)

(73) Assignee: IMPERIAL INNOVATIONS LIMITED, London (GB)

(21) Appl. No.: 14/008,490

(22) PCT Filed: Mar. 28, 2011
(86) PCT No.: PCT/GB2011/000439

§ 371 Date: Dec. 12, 2013

(51) Int. Cl. G01N 33/28

(52) U.S. Cl. CPC G01N 33/2835
USPC 73/61.41

(57) ABSTRACT

An experimental test rig for simulating and analyzing the fouling behavior of petrochemicals when heated within a pipe or other such conduit, the rig comprising: a supply of petrochemical product; an annulus test section arranged to receive petrochemical product from the said supply, the annulus test section comprising an inner tube and an outer tube with the petrochemical product passing therebetween; a tubular test section arranged to receive petrochemical product from the said supply; a pipe circuit connecting the supply of petrochemical product to the test sections; means for heating a region of the annulus test section; means for heating a region of the tubular test section; and means for controlling and measuring the flow of the petrochemical product from the supply to the test sections; wherein the pipe circuit is configured such that the petrochemical product can be simultaneously supplied to the annulus and tubular test sections while they are heated.

### CROSS-REFERENCE TO RELATED APPLICATION

This patent application is the national entry of International Patent Application No. PCT/GB2011/000439, filed on Mar. 28, 2011, entitled “Test Rig and Method for Simulating and Analysing Petrochemical Fouling,” which is assigned to the assignee of the present invention and which is hereby incorporated herein by reference in its entirety.

### BACKGROUND OF THE INVENTION

### Field of the Invention

This invention relates to an experimental test rig and a corresponding method for simulating and analyzing the fouling behavior of petrochemicals when heated within a pipe or other such conduit. It is particularly applicable, but by no means limited, for simulating and analyzing the fouling behavior of crude oil within a heat exchanger, such as in a preheat train in an oil refinery, for example.

Fouling of pipes and conduits by petrochemicals at high temperature is a significant problem in oil refineries and processing plants.

About 6% of the energy content of each crude barrel processed in an oil refinery is used in the refinery itself. Crude oil distillation, where the incoming crude is first heated up and split into its main fractions, accounts for a large proportion of this energy. Thus, strenuous attempts are being made to recover as much as possible of the energy from the product streams of the crude distillation column (and other refinery units) by means of a network of heat exchangers, often called the “pre-heat train” (PHT). Unfortunately, crude oil contains a variety of substances which tend to deposit as fouling layers in the heat exchangers when heated. The material deposited ranges from gel-like to solid-like, and may change its properties with time.

Over time, the growth of the fouling deposit results in decreased energy recovery and thus increased energy demand, with extra cost of fuel and carbon dioxide emissions. Occlusion of tubes in the heat exchangers due to fouling requires extra pumping power to overcome the pressure drops. When the furnace reaches its maximum capacity (firing limit) the crude oil throughput must be throttled back, with serious economic impact. Periodically, individual heat exchangers must be taken out of service and cleaned, again with high impact on productivity, and also considerable health and safety issues.

The economic cost of crude oil fouling in refinery preheat trains is huge. In the US alone it was estimated at around US$1.2 billion per annum (circa 1992), at a time when extra carbon dioxide emissions were not costed. The cost of preheat train fouling in one 160,000 barrels/day refinery was estimated in 2003 to be US$1.5 million in a 3 month period. One 200,000 barrels/day refinery in the UK reported recently (April 2009) that 1° C. of loss of preheat (that is, in the oil temperature at the furnace inlet) cost the operator some £250,000 per annum. Other estimates state that the energy equivalent of some 0.25% of all oil production is lost to fouling in the preheat train. This is equivalent to close to 1 day's production lost per annum (85 million barrels on a worldwide basis).

The principal benefit to the refiners of reduced fouling is increased capacity. Increasing effective on-stream time due to reduced fouling/cleaning can lead to massive savings in some refineries.

The impact of crude oil fouling is increasing for all oil companies. Crudes are generally becoming heavier and more complex, yet refineries were generally designed to process the lighter crudes that are today becoming scarcer. The worldwide shortage of middle distillates is also a driver to the processing of heavier, dirtier crudes that have a higher yield of these valuable components. Fouling problems are therefore increasing in severity.

Fouling by asphaltenes, waxes and hydrates also has serious implications in areas other than refining.

Although progress has been made in experimental studies of crude oil fouling in recent years, it appears that an asymptotic state of knowledge has been reached. In particular, the mechanisms by which the fouling proceeds are still not fully understood. For example, it is generally assumed that materials depositing on heat transfer surfaces are asphaltene derived. These are typically complex mixtures of polynuclear aromatic ring structures, with high heteroatom content; they carry most of the trace element content of the oil. However, it is apparent that only small fractions of the asphaltene content in crudes actually deposit on heat exchange surfaces and that this process can be initiated by other species. There are complex, ill-understood interactions between oil properties, phase stability, rheology, chemical reactions, heat transfer, interfacial and adhesion properties, surface properties and exchanger geometry, all of which affect the fouling mechanisms and sub-processes.

In light of the above discussion, there is clearly a need for a means, within a lab, of simulating and analyzing the fouling behavior of petrochemicals when heated within a pipe or other such conduit, so that the fouling behavior can be better understood, and so that the plant equipment and process parameters in an oil refinery, and their operation, can then be modified, monitored and controlled and the degree of fouling reduced.

The subject matter discussed in this background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

### SUMMARY OF THE INVENTION

According to a first aspect of the present invention there is provided an experimental test rig as defined in Claim 1 of the appended claims. Thus there is provided an experimental test rig for simulating and analyzing the fouling behavior of petrochemicals when heated within a pipe or other such conduit, the rig comprising: a supply of petrochemical product; an annulus test section arranged to receive petrochemical product from the said supply, the annulus test section comprising an inner tube and an outer tube with the petrochemical product passing therebetween; a tubular test section arranged to receive petrochemical product from the said supply; a pipe circuit connecting the supply of petrochemical product to the test sections; means for heating a region of the annulus test section; means for heating a region of the tubular test section; and means for controlling and measuring the flow of the petrochemical product from the supply to the test sections; wherein the pipe circuit is configured such that the petrochemical product can be simultaneously supplied to the annulus and tubular test sections while they are heated.

This apparatus enables lab-based researchers to carry out a series of proving tests on crude oil fouling in situations simulating the conditions in the preheat train of a crude distillation unit. By analyzing the fouling behavior of the petrochemical product within the annulus and tubular test sections simultaneously, this enables direct and meaningful comparisons to be made between the two test sections, using the same supply of petrochemical fluid.

Preferable, optional, features are defined in the dependent claims.

Thus, preferably the petrochemical product is crude oil. This enables a representative simulation to be carried out on the fouling that is experienced in oil refineries.

Preferably the petrochemical product is supplied to the annulus and tubular test sections at high pressure, for example between 10 barg and 30 barg (i.e. between 10 bar gauge pressure and 30 bar gauge pressure), such as approximately 20 barg (i.e. 20 bar gauge pressure). Such pressures are typical of those used in oil refineries, and thus contribute to the authenticity of the simulation.

Preferably the petrochemical product is recirculated around the pipe circuit. This conserves the petrochemical product used, and enables the rig to be operated in a stand-alone, self-contained manner.

Preferably the quantity of petroleum product is approximately 500 liters. This is a sufficiently large quantity to provide meaningful simulation results.

Preferably the said regions of the test sections are heated using Joule heating (i.e. by passing a high current through the walls of the test sections). The use of a Joule heating scheme allows precise control and measurement of the heat flux and also allows access to the metal surface for accurate temperature (and hence heat transfer coefficient) measurement.

Preferably the Joule heating is applied to the said regions of the test sections using metal clamps configured to fit tightly around the said regions. Such clamps ensure that electrical losses due to gap resistances are minimized, thereby improving the delivery of the Joule heating current to the said regions, and improving the efficiency and control of the heating process.

Particularly preferably the metal clamps are made of copper, as this has high electrical conductivity.

Preferably the metal clamps incorporate conduits therethrough, to provide a flow of cooling liquid in use. By passing a flow of cooling liquid through the clamps while they are causing the test sections to be heated, this ensures that the clamps themselves do not become excessively hot.

Preferably the said regions of the test sections are electrically isolated from the rest of the test sections. This confines the Joule heating current to the intended regions of the test sections, and prevents Joule heating current from leaking elsewhere.

The said regions of the test sections may be heated to a temperature in the range of 250-350° C., for example approximately 300° C., which are representative of temperatures experienced in oil refineries.

Preferably the rig further comprising means for measuring the temperature of the heated region of the annulus test section. Particularly preferably this is measured by a thermocouple inside the inner tube of the annulus test section. Positioning the thermocouple inside the inner tube of the annulus test section enables the wall temperature of the inner tube to be measured in a number of places, and prevents the thermocouple from obstructing or being fouled by the fouling deposit which builds up on the outside of the inner tube.

In the presently-preferred embodiment the thermocouple is a radiation equilibrium thermocouple, which is preferably mounted on a motorized (servo-assisted) traverse mechanism to enable the temperature to be measured at a number of accurately-defined points along the annulus test section, with a high degree of repeatability.

Preferably the said thermocouple is contained within a ceramic shield having a small window. This ensures that the temperature measurement is extremely localized, and also minimizes heat losses around the thermocouple tip.

Preferably the ceramic shield is shaped to fit closely within the inner tube of the annulus test section, so as to minimize heat losses around the thermocouple tip.

Preferably the rig further comprising means for measuring the temperature of the heated region of the tubular test section. Particularly preferably this is measured by a series of thermocouples.

Preferably the rig further comprising a cooling circuit arranged to remove heat added to the petrochemical product in the heated test sections.

Preferably the cooling circuit comprises a first heat exchanger arranged to transfer heat from the petrochemical product to a first (preferably non-aqueous) heat transfer fluid. The use of a non-aqueous heat transfer fluid in this manner enables controlled heat removal from the petrochemical product at a higher temperature than would be the case if direct heat transfer to a cooling water stream were used.

Preferably the cooling circuit further comprises a second heat exchanger arranged to transfer heat from the first heat transfer fluid to a second heat transfer fluid, which is preferably water. The use of this second heat exchanger in this manner provides effective cooling of the first heat transfer fluid. Together the first and second heat exchangers enable the temperature of the petrochemical product to be accurately controlled, as they enable the heat added in the test sections to be removed while maintaining a relatively high temperature overall, i.e. without excessive or unnecessary cooling and reheating.

Preferably the test sections are at least partly made from carbon steel, and substantially the rest of the pipe circuit is made of stainless steel. The use of carbon steel for the fouling surfaces in the test sections simulates the real fouling conditions in an actual oil refinery, whereas the use of stainless steel in substantially the rest of the pipe circuit helps to reduce oxidation, corrosion or fouling elsewhere.

Preferably the test sections are removable from the rest of the pipe circuit, to enable them to be taken away and the fouling deposits examined.

Preferably the rig further comprises a container in which the supply of petrochemical product, the test sections and the pipe circuit and are housed, the container being flooded with nitrogen. This helps to maintain safe operation of the rig.

Preferably the rig is mounted on wheels, to enable it to be moved from one location to another. This allows the rig to be set up in alternative locations, for example in a lab, or in an oil refinery where direct measurements on the crude oil being utilized can be made.

According to a second aspect of the present invention there is provided a method of simulating and analyzing the fouling behavior of a petrochemical product, using a rig in accordance with the first aspect of the invention, the method comprising: heating the said regions of the annulus and/or tubular test sections; causing the petrochemical product to flow through the annulus and/or tubular test sections; and measuring the fouling of the test sections by the petrochemical product.

Preferably the method comprises heating the said regions of the annulus and tubular test sections simultaneously, and causing the petrochemical product to flow through the annulus and tubular test sections simultaneously.

Preferably the method further comprises monitoring the temperature of the heated region(s) of the test section(s) over time. Particularly preferably the method further comprises determining the temperature of the surface of the heated region(s) in contact with the flowing petrochemical product over time.

Preferably the method further comprises monitoring the heat transfer rate between the heated region(s) of the test section(s) and the flowing petrochemical product over time. This can then be used to determine the degree/evolution of fouling as a function of time.

The method may further comprise monitoring the pressure drop in the test sections(s) over time. The pressure drop data can then be used to determine the degree/evolution of fouling as a function of time.

Finally, the method may further comprise removing the test section(s) from the rig and examining it/them, to investigate the fouling deposits therein.

With both the aspects of the invention, preferable, optional, features are defined in the dependent claims.

### DESCRIPTION OF THE DRAWINGS

Embodiments of the invention will now be described, by way of example only, and with reference to the drawings in which:

FIG. 1 is a schematic flow sheet of a test rig in accordance with an embodiment of the invention;

FIG. 2a illustrates design detail (isometric and plan views) of a copper clamping arrangement to supply Joule heating to a test section of the rig;

FIG. 2b illustrates the copper clamping arrangement of FIG. 2a in place on an annulus test section of the rig;

FIG. 3a illustrates design detail of a shield for a thermocouple tip for a radiation equilibrium thermocouple system;

FIG. 3b is an exploded diagram of the shield of FIG. 3a;

FIG. 3c illustrates the shield of FIG. 3a attached to a tube containing the thermocouple, removed from the annulus test section;

FIG. 4 illustrates the apparatus of FIG. 3c mounted on a traversing drive mechanism, in retracted and inserted positions relative to an annulus test section;

FIG. 5 shows more detail of the drive mechanism for the radiation equilibrium thermocouple system of FIG. 4;

FIG. 6 is a schematic overview of a control and data acquisition system for the rig;

FIG. 7 is a flow-sheet mimic for control of the rig; and

8 illustrates detail of a wall mounted thermocouple configuration used in the rig.

In the figures, like elements are indicated by like reference numerals throughout.

### DETAILED DESCRIPTION OF AN EXEMPLARY EMBODIMENT

The present embodiment represents an exemplary way known to the applicants of putting the invention into practice. However, it is not the only way in which this can be achieved.

### Overview

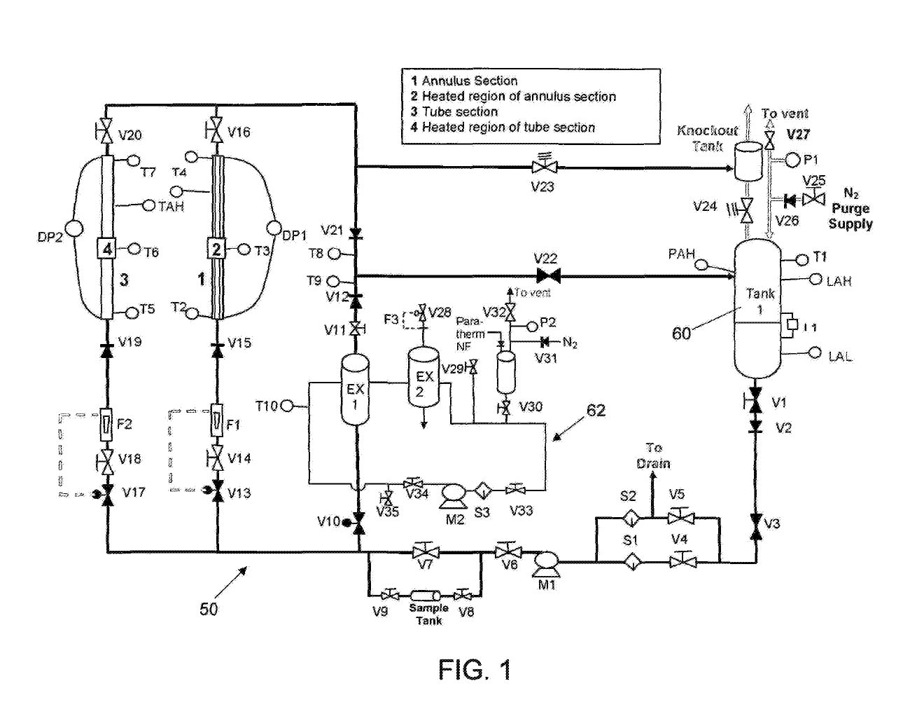

As illustrated in FIG. 1, the presently preferred embodiment is a test rig 50 (which is normally but not necessarily lab-based) comprising a high temperature, high pressure, crude oil flow loop equipped with an annulus test section 1 and a tubular test section 3, each having a heated region 2, 4 respectively on which fouling deposits will form. A complete list of the numbered components which make up the rig 50 in FIG. 1 is provided in Appendix 1. The rig 50 offers important benefits including the accurate measurement of heat transfer behavior through the use of Joule heating and special methods for wall temperature measurement; the simultaneous measurement of fouling in the tubular and annulus test sections 3, 1; and the ability to handle sufficient volumes of crude oil at appropriate temperatures and pressures in order to give a representative simulation of refinery conditions.

The present rig is primarily for use in studying the effects of fouling on heat transfer and pressure drop in Joule-heated test sections, simulating flow in a shell-and-tube heat exchanger. Two types of test section are available on the rig, namely an annulus test section 1 and a tubular test section 3; however, the rig has sufficient flexibility to accommodate other test configurations as required. For instance, the rig can be modified to study fouling in more complex heat exchanger geometries or by adding inserts within the test tube.

The annulus and tubular test sections 1, 3 in the present rig each have a high current, low voltage power supply for Joule heating. Here, “Joule heating” means heating the specific test section regions 2, 4 respectively by passing a high current through their walls. Such a heating scheme allows precise control and measurement of the heat flux and also allows access to the metal surface for accurate temperature (and hence heat transfer coefficient) measurement.

The tubular test section 3 is typically a sample of carbon steel heat exchanger tubing; in this case, the crude oil flows in the inside of the tube and wall temperature is measured on the outside of the tube using a series of fixed thermocouples (for example as illustrated in FIG. 8).

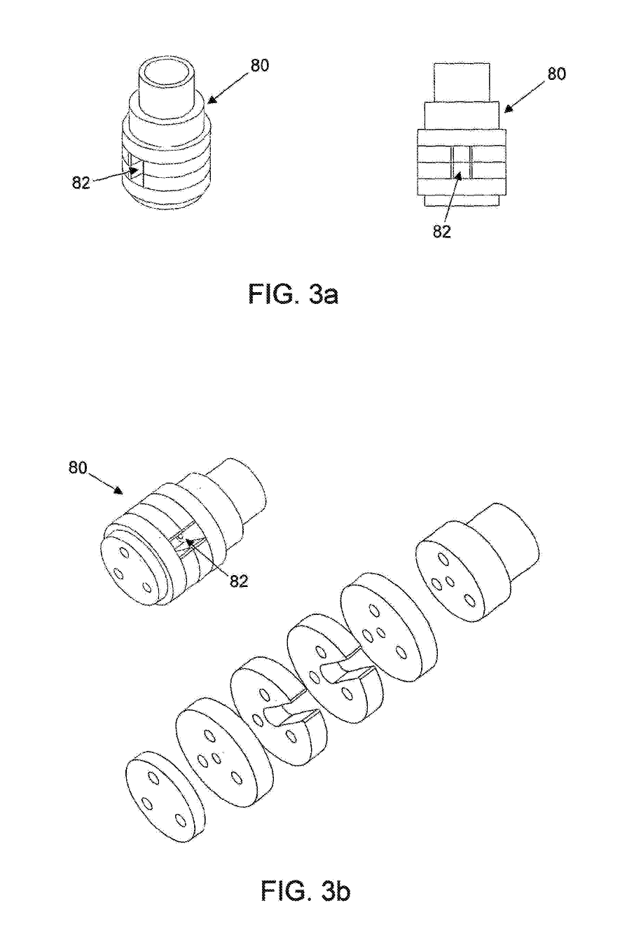

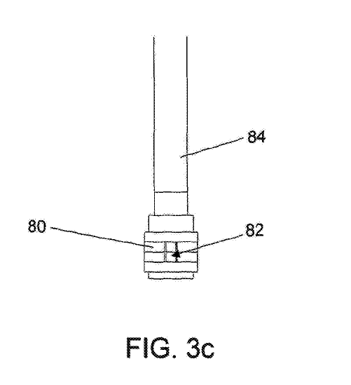

The annulus test section 1 consists of a heated inner carbon steel tube with the crude oil flowing between this tube and an unheated stainless steel outer tube. For the annulus test section, accurate monitoring of the inner surface temperature of the inner (heated) tube is achieved using a traversable radiation equilibrium thermocouple inside the inner tube. This is described in greater detail in section 3 below, with reference to FIGS. 3a, 3b, 3c, 4, and 5.

Both the tubular and annulus test section configurations permit the continuous monitoring of the heat transfer rate between the electrically heated test section surface and the flowing crude oil, and the accurate estimation of the temperature of the surface in contact with the crude oil. Thus, the heat transfer coefficient between the surface and the crude oil can be continuously monitored and the fouling resistance determined as function of time. In both cases, the pressure drop in the test section is also monitored and the effect of fouling on pressure gradient can be evaluated.

While, at first sight, the tubular test section 3 may appear to be more directly relevant to the case of tube-side flow in a shell-and-tube heat exchanger, the use of the annulus test section 1 with fouling on the outside of the inner tube offers several important advantages as follows:

a) The wall temperature can be determined at all points along the heated surface by use of the traversing radiation equilibrium thermocouple system inside the inner tube.
b) The test section can be removed after the test and the thickness of the fouling layer measured and the material in the layer sampled and analyzed.
c) The use of an annulus test section allows the application of a thickness sensor for the continuous in situ measurement of fouling layer thickness.

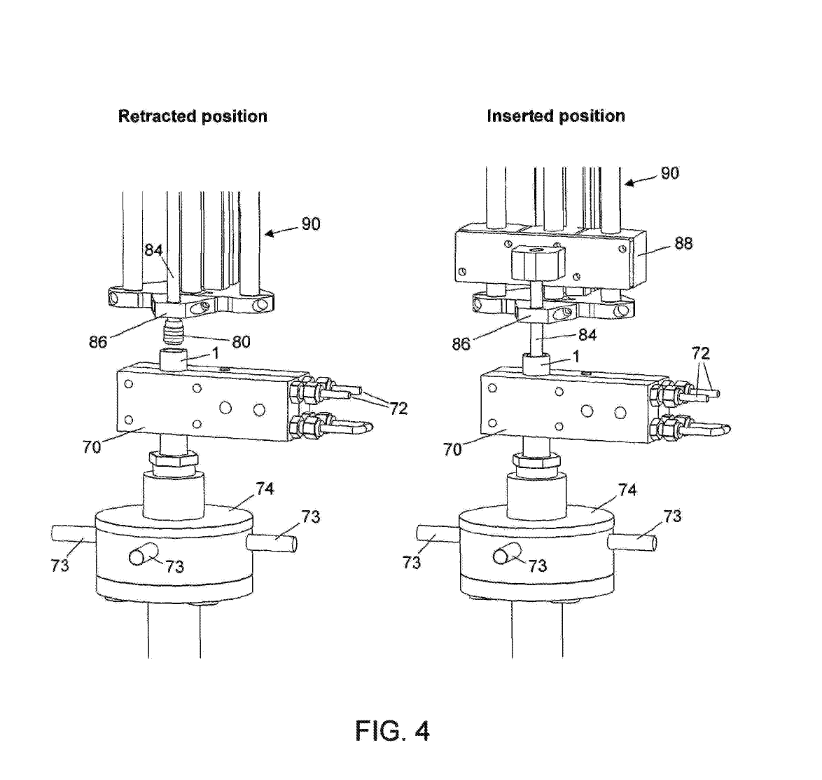

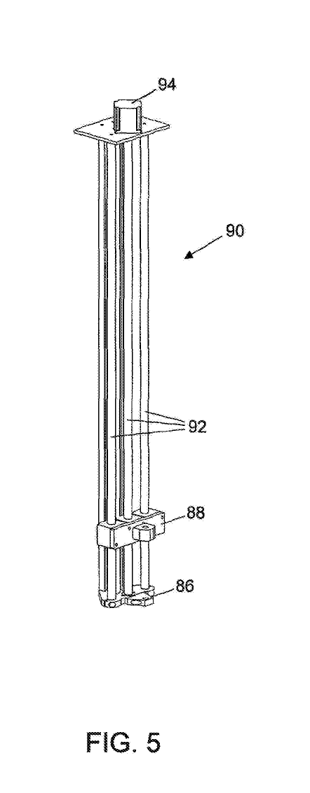

The two test sections 1, 3 can be simultaneously run in parallel or in series. As illustrated in FIG. 1, they are preferably run in parallel. For the annulus test section 1, the temperature of the inner wall of the heated tube is measured using a radiation equilibrium thermocouple which is contained within ceramic shields (FIGS. 3a, 3b, and 3c), and which traverses axially down the test section (FIGS. 4 and 5) and measures the temperature of the inner wall of the heated tube to a high degree of accuracy. The temperature of the outer wall of the inner tube, on which the fouling deposition occurs, can be calculated from this measured inner wall temperature (for the known electrical power input) by consideration of the radial conduction through the cylindrical tube wall. The outer wall temperature changes, at a given power input and oil flow rate and inlet temperature, in response to build-up of the fouling layer on the tube outer surface. The heat transfer measurements, and measurements of the thickness of the fouling layer, can be used to derive an estimation of the thermal conductivity of the fouling layer. The pressure gradient is measured using high temperature pressure transducers. Since the surface of the deposit will be rough and the outer tube relatively smooth the shear stresses on the two surfaces will be different; however, the (very important) shear stress on the inner surface may be calculated from the pressure gradient using a transformation method and flow rate information.

The crude oil sample (approximately 500 liters) is stored in a nitrogen-pressurized high pressure, high temperature vessel 60 and is pumped from this vessel to the test sections (indicated as 1 for the annulus section and 3 for the tubular test section; the heated zones are labeled 2 and 4 respectively).

Heat added to the fluid in its passage through the test sections 1, 3 is removed in a heat exchanger EX1, the heat being discharged to a non-aqueous heat transfer fluid (e.g. PARATHERM®, or some other non-aqueous heat transfer liquid) which flows in a secondary loop 62 on the cold side of EX1. In turn, the PARATHERM® is cooled in a further exchanger EX2, where the cooling water flows on the cold side of the exchanger. The main flow loop is designed to supply crude oil to the heated test sections 1, 3 at a maximum temperature of 300° C. The maximum loop operating pressure is 30 barg (i.e. 30 bar gauge pressure). The annulus test section 1 is 2 m long, permitting fouling measurements over about 1.5 m, after allowance for an entry section for flow stabilization. The optimal oil delivery configuration (4 injection points) was identified from CFD (Computational Fluid Dynamics) studies. Test surface temperatures of 250-350° C. are feasible, i.e. oil plus 120° C. maximum, leading to an imparted heat flux from the surface to the oil in the region 20-100 kW m⁻². The fluid hydrodynamics will be representative of conditions in refinery shell-and-tube heat exchangers, with a linear velocity in the region 1-3 m/s, and Reynolds number up to a maximum of 20,000. The tubular test section 3 operates over a similar range of conditions.

In operation with crude oils, the whole of the rig 50 is contained in a metal box. This box is purged with a nitrogen stream (derived from a pressure-swing adsorption nitrogen generator) and this nitrogen stream is discharged to atmosphere outside the laboratory. The crude oil from the vessel 60 is recirculated around the pipe circuit.

Some of the above features will now be described in greater detail:

### 1. The Use of Joule Heating

This involves the heating of the test sections 1, 3 by passing large electrical currents through their walls, thus generating heat in the wall in a manner which can be precisely controlled and measured. Joule heating also facilitates the accurate measurement of the surface temperature of the metal in contact with the fouling layer and hence of the fouling resistance.

In the design of the Joule heating system, special attention was given to the design of the current clamps which transmit the current from the power supply to the test section. This involved the design of a special cooling system, reflecting the very high temperatures encountered in the crude oil fouling tests.

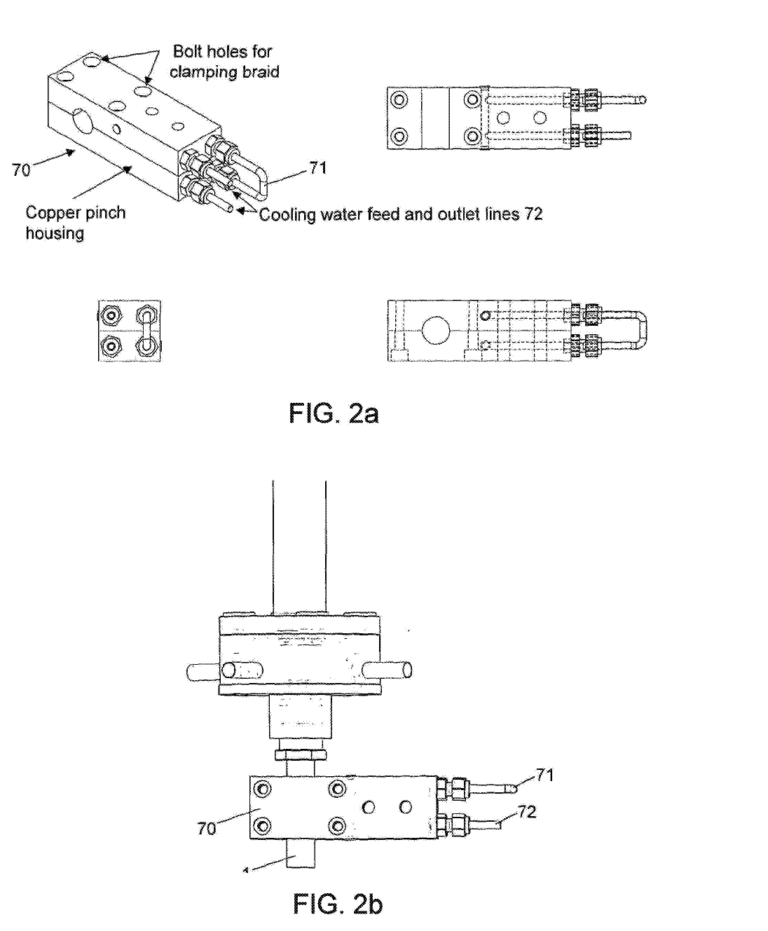

As shown in FIGS. 2a and 2b, the copper clamping apparatus 70 is specifically designed to provide a tight fit on the tubular section to be heated by using a pinch housing arrangement. This arrangement also allows the copper braid providing the electrical current to be tightly connected. This ensures that the electrical losses due to gap resistances are minimized. Since the main test sections are heated to high temperatures (˜300° C.) an additional piping manifold has been implemented in the clamping arrangement to enable a feed 72 of cooling liquid to pass through the clamp 70. This ensures that the clamp 70 does not become excessively hot while it causes the corresponding test section to be heated.

### 2. The Use of Simultaneous Testing in Tubular and Annulus Test Sections

The tubular test section 3 is more typical of a real heat exchanger but the annulus test section 1 is better from the point of view of observing and measuring the characteristics of the fouling layer. The simultaneous use of both geometries allows the relevance of the (more flexible) annulus arrangement to be confirmed. The inner (heated) surface in the annulus can be removed and observation made of the fouling layer without the need for destroying the test section as would be the case with a tube. The annulus test section 1 also allows access for probes for continuous on-line measurement of the fouling layer thickness.

The design of the rig is such that the flow areas of the tubular and annular sections 3, 1 are similar, so that direct comparisons can be made. The annulus section 1 has the advantage that the central (heated) surface can be removed non-destructively from the rig and the fouling layers examined in detail using various analytical techniques. Specifically, in the case of the annulus, the thickness of the fouling layer on the heated surface can be readily determined. A very important feature of the rig is that the same fluid passes both though both the annulus and tubular test sections so direct and meaningful comparisons can be made between the two. The flow to each of the test sections is controlled and monitored separately. In the case of the annulus test section 1, a manifold design was developed (using Computational Fluid Dynamics) which meets the requirements of uniformity of flow at the entrance to ensure uniformity of the behavior of the entire surface of the annulus.

### 3. The Use of Radiation Equilibrium Thermometry for the Annulus Test Section

Temperature measurement within the annulus test section 1 is made using a radiation shielded thermocouple which can be traversed up and down the (empty) inside of the inner tube of the annulus test section 1. Experiments on boiling heat transfer have demonstrated that this allows a very accurate measurement of the inner wall temperature, from which the temperature of the outer surface in contact with the oil can be calculated.

The radiation equilibrium thermocouple system is specially designed for the present rig. Special features of the present design include the specific structure for the tip of the thermocouple probe (FIGS. 3a, 3b, and 3c), which is designed to provide directional localized measurements of the inside surface temperature of the heated tube in the annulus test section 1. Another noteworthy feature of the radiation equilibrium thermocouple system for the annulus section 1 is a servo-assisted traversing mechanism 90 (FIGS. 4 and 5) which was also specially designed for the present rig, enabling the thermocouple to be accurately moved to different positions along the annulus test section 1. This positioning device is designed to operate in an inert atmosphere and is capable of positioning the radiation equilibrium thermocouple within the central annulus tube to a high degree of accuracy (the specific design allows the thermocouple to be placed within ±0.5 mm).

FIGS. 3a, 3b, and 3c show the design of the shielding 80 for the thermocouple tip within the annulus test section 1. As illustrated in FIG. 3b, the shielding 80 may be constructed from a number of layers of insulating material—for example KLINGERSIL® C-4400, or any other suitable insulating material as will be apparent to those skilled in the art. The insulating material is assembled so that the thermocouple tip is only exposed through a small window 82 in the shield 80. This ensures that the temperature measurement is extremely localized. The insulating shield 80 is also designed to fit closely within the inner annulus section 1 of the flow loop, so as to minimize heat losses around the thermocouple tip. FIG. 3c shows the shielding 80 attached to a tube 84 containing the thermocouple. The thermocouple tip is positioned behind the window 82.

FIG. 4 illustrates the construction and operation of the traversing mechanism 90. The traversing mechanism 90 comprises a centralizing tube guide 86 and a drive 88 for moving the tube 84 containing the thermocouple, which is shown in retracted and inserted positions relative to an annulus test section 1. With reference to FIG. 5, the drive 88 is moveably mounted on three guide rods 92 and is driven by a servo motor 94, so that the thermocouple tip can be accurately positioned at various locations along the annulus test section 1 with a high degree of repeatability.

FIG. 4 also shows the water cooled clamping system 70, 71, 72 in situ. This comprises one line for the cooling water feed (marked 72) into the clamping arrangement 70 on one side, a U-shaped connection 71 between the two halves (lower down), and then a cooling water return (also marked 72) on the other half of the clamp 70.

The cylindrical chamber 74 below the water cooled clamp is a manifold designed to enable oil to exit smoothly from the annulus test section 1 while allowing the heated inner tubular section freedom to expand or contract due to thermal changes and still maintain electrical contact to the power supply.

There is a similar manifold 74 at the inlet to the annulus test section 1. The inlet manifold has four individual feed lines 73 so that an even distribution of the fluid feeding into the annular gap can be produced within the annulus test section 1. Computational Fluid Dynamic (CFD) simulations of this configuration indicate that a fully developed profile (both velocity and thermal) can be attained within a relatively short distance from the point of introduction of the fluid.

### 4. The Use of a Secondary (Heat Transfer Fluid) Circuit to Remove the Heat Added to the Crude Oil Stream

The secondary circuit (62 in FIG. 1) allows controlled heat removal from the oil at a higher temperature than would be the case if direct heat transfer to a cooling water stream were used.

It is important to cool the crude oil down to remove heat added in the test sections. However, it is also important to maintain the high temperature of the crude oil without excessive cooling and reheating. In order to achieve this, the secondary circuit 62 uses an intermediate heat transfer fluid (PARATHERM®) which allows the crude oil to be kept at a high temperature while removing the added heat. Finally, heat is removed from the PARATHERM® using a conventional PARATHERM/water heat exchanger EX2. This two-stage heat removal process is important in the control and design of the equipment.

### 5. Portability

The whole of the present rig is designed such that it can be moved on a wheeled “skid” from one location to another. This aspect of the design allows the facility to be set up in alternative locations, for example in an oil refinery where direct measurements on the crude being utilized at any given time can be made.

### 6. Control System

A specific and special control system has been designed to operate the rig to meet the operational goals. Control over the flow rates, crude oil temperature, cooling system temperature and heat flux inputs is organized through new computer code which is embodied in a controlled computer on the facility. Additional software has been developed to control the position of the radiation equilibrium thermocouple for the annulus test section 1 so that continuous metering of the test section temperatures can be achieved over long periods of time.

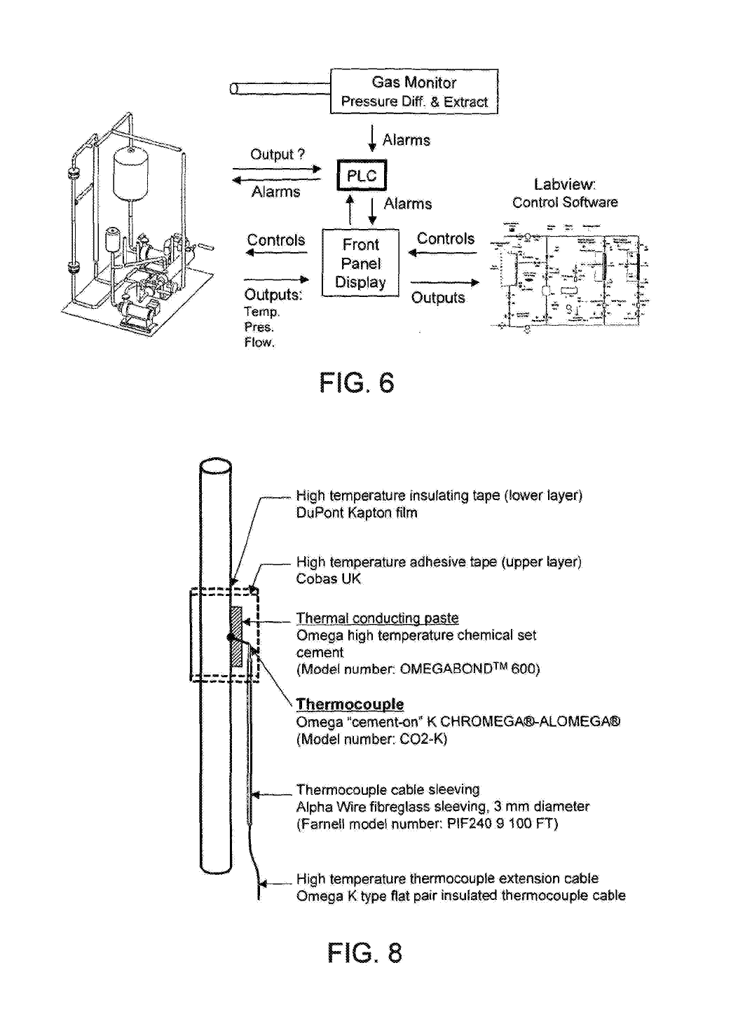

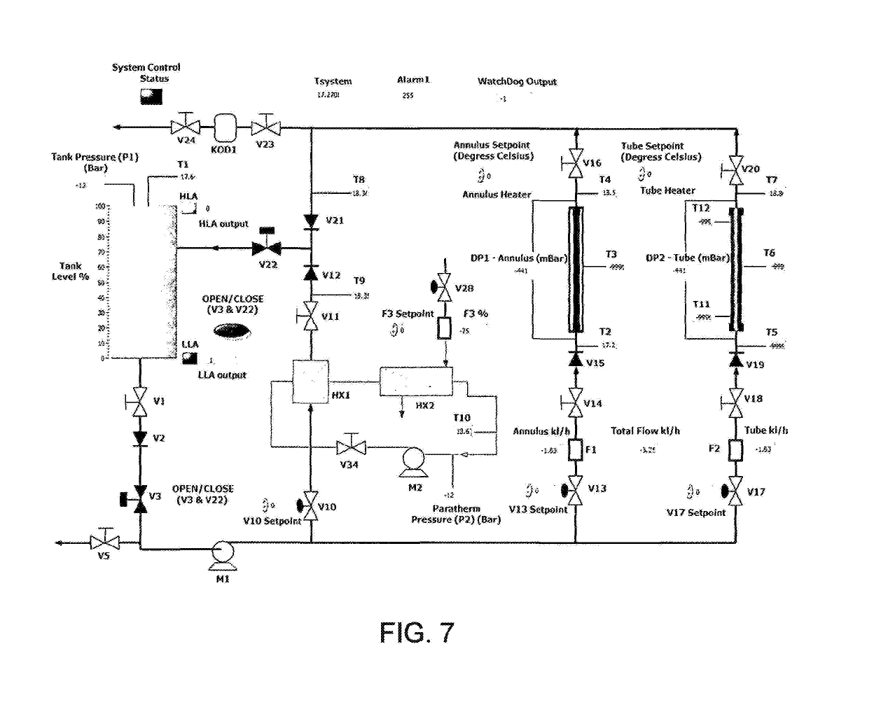

The control and data acquisition system for the rig is illustrated in FIGS. 6 and 7. FIG. 6 shows an overview of the control system. Basically, three specific functions are integrated into this system. These are as follows:

a) High pressure control system (the main rig).
b) PLC monitoring system (safety alarms and cut-offs).
c) Environment control system (containment box and surrounding area).

FIG. 7 shows a flow-sheet mimic used to control the present rig. From this screen, key valves such as V3 and V22 may be opened and closed to enable the main feed tank 60 to be isolated. Also, the set-points on control valves V10, V13, and V17 may be specified. The heaters (and set-point temperatures) on both the annulus and tubular sections 1, 3 may also be set from this flow-sheet. In addition to these operations the flow-sheet also provides a real time display of the key process parameters such as pressures, temperatures and flow rates. These parameters may be simultaneously logged to a separate file for subsequent data analysis.

### 7. Maintaining Safe Operation

An entirely new system for maintaining safe operation on the facility has been designed. This involves putting the whole of the working circuits of the facility inside a container which is flooded with nitrogen. Measurements of the hydrocarbon content in the container are made to ensure that no hydrocarbon is escaping from the circuit. Also, measurements of oxygen content in the container are made and this indicates whether the atmosphere is at a suitable composition to prevent fire arising in the case of a leak. On the outside of the container, measurements are made of hydrogen sulphide (which is a component of some crude oils) to ensure personnel safety, as are measurements of oxygen composition to ensure that the local concentrations of nitrogen do not raise significantly above atmospheric values.

### 8. Selection of Materials

The main circuit components are made of stainless steel to prevent oxidation or corrosion. Only those parts of the test sections 1, 3 which are heated and are intended to simulate the fouling surfaces are made of carbon steel (thus simulating the real situation in a crude oil heat exchanger). If stainless steel had not been used elsewhere in the circuit, the whole circuit would have a propensity to foul (as in the case of carbon steel) and this would make the rig non-operational. This choice of materials ensures that asphaltene deposits only occur on the carbon steel test sections 1, 3. In turn, this should ensure that the measurements of deposition rates etc. are more reliable.

### 9. Electrical Isolation Around the Joule Heated Sections

Special systems have been developed for electrical isolation of the annulus and tubular test sections 1, 3. Since the test sections 1, 3 are within a metallic circuit and are directly heated by Joule heating, it is important that the heated regions 2, 4 respectively are electrically isolated from the rest of the flow loop circuit, to prevent leakage of the Joule heating current. This is achieved using special sealing materials. Electrically insulating gaskets are provided between flanges either side of the heated regions 2, 4. The gaskets are also able to withstand the high temperatures that they are likely to be subjected to.

For this purpose, in our current prototype, KLINGERSIL® C-4400 gaskets are placed between raised face flanges either side of the heated regions 2, 4. To ensure that there is no electrical path between the flanges due to the retaining bolts, KLINGERSIL® C-4400 washers have also been constructed, and the bolts are sleeved with a non-conducting tape (Cobas UK).

To ensure that there is no conducting path between the cooling water connections to the electrical clamps fitted to each test section and the rest of the flow loop, dielectric fittings are used (HAMLET 762LSSl/4-Dielectric).

Naturally it will be appreciated that alternative materials may be used for the gaskets, washers and other components that are provided to electrically isolate the Joule heated test sections.

FIG. 8 shows the details of a prototype arrangement used to ensure that the wall mounted thermocouples associated with the tubular test section 3 are able to measure the wall temperature while being electrically isolated from the tubular test section 3. Again, materials and components other than the specific ones identified may be used, as those skilled in the art will appreciate.

Although the foregoing description of the present invention has been shown and described with reference to particular embodiments and applications thereof, it has been presented for purposes of illustration and description and is not intended to be exhaustive or to limit the invention to the particular embodiments and applications disclosed. It will be apparent to those having ordinary skill in the art that a number of changes, modifications, variations, or alterations to the invention as described herein may be made, none of which depart from the spirit or scope of the present invention. The particular embodiments and applications were chosen and described to provide the best illustration of the principles of the invention and its practical application to thereby enable one of ordinary skill in the art to utilize the invention in various embodiments and with various modifications as are suited to the particular use contemplated. All such changes, modifications, variations, and alterations should therefore be seen as being within the scope of the present invention as determined by the appended claims when interpreted in accordance with the breadth to which they are fairly, legally, and equitably entitled.

While the current application recites particular combinations of features in the claims appended hereto, various embodiments of the invention relate to any combination of any of the features described herein whether or not such combination is currently claimed, and any such combination of features may be claimed in this or future applications. Any of the features, elements, or components of any of the exemplary embodiments discussed above may be claimed alone or in combination with any of the features, elements, or components of any of the other embodiments discussed above.

APPENDIX 1
Test rig components in FIG. 1

| Part symbol | Description |
|---|---|
| 1 | Annulus section |
| 2 | Heated region of |
| | annulus section |
| 3 | Tube section |
| 4 | Heated region of |
| | tube section |
| T1-T10 | Thermocouple |
| VI, V4, V5, V6, V7, V8, V9, V11, | Manual valve |
| V14, V16, V18, V20, V25, V29, | |
| V30, V33, V34, V35 | |
| V2, V12, V15, V19, V21, V26, V31 | Check valve |
| V10, V13, V17, V27, V28, V32 | Control valve |
| V3, V22 | Isolation valve |
| V23, V24 | Relief valve |
| SI, S2, S3 | Strainer |
| M1, M2 | Pump |
| EX1, EX2 | Heat exchanger |
| P1, P2 | Pressure transducer |
| DP1, DP2 | Differential |
| | pressure transducer |
| F1, F2 | Flow meter |

1. An experimental test rig for simulating and analyzing the fouling behavior of petrochemicals when heated within a pipe or other such conduit, the rig comprising:
a supply of petrochemical product;
an annulus test section arranged to receive petrochemical product from the supply, the annulus test section comprising an inner tube and an outer tube with the petrochemical product passing therebetween;
a tubular test section arranged to receive petrochemical product from the supply;
a pipe circuit connecting the supply of petrochemical product to the test sections;
means for heating a region of the annulus test section;
means for heating a region of the tubular test section; and
means for controlling and measuring the flow of the petrochemical product from the supply to the test sections;
wherein the pipe circuit is configured such that the petrochemical product can be simultaneously supplied to the annulus and tubular test sections whilst they are heated.

2. (canceled)

3. A rig as claimed in claim 1, wherein the petrochemical product is supplied to the annulus and tubular test sections at high pressure, wherein the high pressure is a pressure of between 10 barg and 30 barg, and wherein the high pressure is optionally a pressure of approximately 20 barg.

4-5. (canceled)

6. A rig as claimed in claim 1, wherein the petrochemical product is recirculated around the pipe circuit.

7. (canceled)

8. A rig as claimed in claim 1, wherein the regions of the test sections are heated using Joule heating, and wherein the Joule heating is applied to the regions of the test sections using metal clamps configured to fit tightly around the regions.

9. (canceled)

10. A rig as claimed in claim 8, wherein the metal clamps are made of copper, and wherein the metal clamps incorporate conduits therethrough, to provide a flow of cooling liquid in use.

11. (canceled)

12. A rig as claimed in claim 8, wherein the regions of the test sections are electrically isolated from the rest of the test sections.

13. A rig as claimed in claim 1, wherein the regions of the test sections are able to be heated to a temperature in the range of 250-350° C., and wherein the regions of the test sections are optionally able to be heated to a temperature of approximately 300° C.

14. (canceled)

15. A rig as claimed in claim 1, further comprising means for measuring the temperature of the heated region of the annulus test section, wherein the temperature measuring means optionally comprises a thermocouple inside the inner tube of the annulus test section, and wherein the thermocouple optionally comprises a radiation equilibrium thermocouple inside the inner tube of the annulus test section.

16-17. (canceled)

18. A rig as claimed in claim 15, wherein the thermocouple is movably mounted on a traverse mechanism, wherein the thermocouple is optionally movably mounted on a traverse mechanism, and wherein the traverse mechanism is optionally motorized, and wherein the traverse mechanism is optionally servo-assisted.

19-22. (canceled)

21. A rig as claimed in claim 15, wherein the thermocouple is contained within a ceramic shield having a small window, and wherein the ceramic shield is optionally shaped to fit closely within the inner tube of the annulus test section.

23. A rig as claimed in claim 1, further comprising means for measuring the temperature of the heated region of the tubular test section, wherein the temperature measuring means optionally comprises a series of thermocouples.

24. (canceled)

25. A rig as claimed in claim 1, further comprising a cooling circuit arranged to remove heat added to the petrochemical product in the heated test sections, wherein the cooling circuit optionally at least one of a first heat exchanger arranged to transfer heat from the petrochemical product to a first heat transfer fluid and a second heat exchanger arranged to transfer heat from the first heat transfer fluid to a second heat transfer fluid.

26-27. (canceled)

28. A rig as claimed in claim 25, wherein the first heat transfer fluid is a non-aqueous heat transfer fluid, and/or wherein the second heat transfer fluid is water.

29. (canceled)

30. A rig as claimed in claim 1, wherein the test sections are at least partly made from carbon steel, and substantially the rest of the pipe circuit is made of stainless steel, and/or wherein the test sections are removable from the rest of the pipe circuit, and/or wherein the rig may further comprise a container in which the supply of petrochemical product, the test sections and the pipe circuit and are housed, the container being flooded with nitrogen.

31-33. (canceled)

34. A method of simulating and analyzing the fouling behavior of a petrochemical product, using a rig as claimed in any preceding claim, the method comprising:
heating the regions of the annulus and/or tubular test sections;
causing the petrochemical product to flow through the annulus and/or tubular test sections; and
measuring the fouling of the test sections by the petrochemical product.

35. A method as claimed in claim 34, further comprising recirculating the petrochemical product through the annulus and/or tubular test sections.

36. A method as claimed in claim 34, comprising heating the regions of the annulus and tubular test sections simultaneously, and causing the petrochemical product to flow through the annulus and tubular test sections simultaneously.

37. A method as claimed in claim 34, further comprising monitoring the temperature of the heated region(s) of the test section(s) over time, and optionally further comprising determining the temperature of the surface of the heated region(s) in contact with the flowing petrochemical product over time, and/or optionally further comprising monitoring the heat transfer rate between the heated region(s) of the test section(s) and the flowing petrochemical product over time, and optionally further comprising using the monitored heat transfer rate to determine the degree of fouling as a function of time.

38-40. (canceled)

41. A method as claimed claim 35, further comprising monitoring the pressure drop in the test sections(s) over time, and optionally further comprising using pressure drop data to determine the degree of fouling as a function of time.

42. (canceled)

43. A method as claimed in claim 35, further comprising removing the test section(s) from the rig and examining it/them.

44-45. (canceled)
